(19) United States
(12) Patent Application Publication
(10) Pub. No.: US 2005/0202524 A1
(43) Pub. Date: Sep. 15, 2005

(54) TISSUE STAINING PROTOCOL AND COMPOSITION

(76) Inventor: Arkadiy I. Brusilovskiy, Santa Monica, CA (US)

Correspondence Address: GREENBERG & LIEBERMAN, 314 PHILADELPHIA AVE., TAKOMA PARK, MD, 20912, US

(21) Appl. No.: 10/906,991

(22) Filed: Mar. 15, 2005

Related U.S. Application Data
(60) Provisional application No. 60/521,225, filed on Mar. 15, 2004.

(51) Int. Cl. G01N 33/48

(52) U.S. Cl. 435/40.5

(57) ABSTRACT

An enhanced and particularly quick histological staining method is expressed in which a frozen tissue or deparafffinized tissue sample is briefly immersed in an eosin solution, used as a first dye, and then in a hematoxylin solution, used as a second die. The slide is rinsed and air-dried to make ready for assessment.

### FIELD OF INVENTION

The present invention generally relates to a novel combination for staining histological slides of cells, tissues, and organs, and more particularly, to a method of staining comprising of a combination where eosin is applied as first dye and hematoxylin is applied as a second dye. More particularly for a combination for staining that will reveal new chemical components and structures in cells, tissue, and organ samples not seen with a conventional hematoxylin and eosin stain.

### BACKGROUND OF THE INVENTION

In order for the scientific community to gain rapid increase in the information of diseases, precise diagnosis is needed to maximize efficiency of treatments. Histology and cytology have been two popular diagnostic techniques that scientists utilize to gain insight into the structures and mechanism of cells and tissues. Both techniques involve the staining of tissue cells to distinguish different parts of the cells. However, these techniques, as presently known, are not with out fault as they are often time consuming and burdensome.

Cytology involves fixing slides in alcohol, staining slides using the Papanicolau method, and then staining air-dried slides with a Romanowski type stain such as giemsa, MGG, or repidiff. While cytology is cheaper and quicker to perform and get diagnostic results than histology methods, the samples produced provide less information and are often easily perishable.

Histology entails examination of solid tissue samples, which are sectioned cut and stained for microscopic examination. Chemicals and stains are applied to sections to emphasize various cells or tissue components, thus providing a visual contrast that aids in identifying specific tissue components and causes diagnosis to be more reliable.

Most staining reactions engage a chemical merger between dye and stained substances through salt linkages, hydrogen bonds, and the like. The stain results are predictable in color pattern based partly on acid base characteristics of the tissue. Most histological dyes classify as acid or as basic dyes. Acid dye exists as an anion, being negatively charged in solution. Basic dye exists as a cation, being positively charged in solution. Moreover, any substance stained by basic dye is basophilic, carrying acid groups, which bond the basic dye through salt linkages. Conversely, any substance stained by acid dye is acidophilic, carrying basic groups, which bind the acid dye through salt linkages. In conventional histology, hematoxylin is applied as a first dye and eosin as a second dye because hematoxylin is a prime basophilic stain and eosin is a prime acidophilic stain. Thus, the results of using these dyes in the staining process of histology produce a tissue specimen with a bluish/purple (acidic “basophilic” components of tissue) and pinkish/red (basic “acidophilic” components of tissue) coloring.

There are two major techniques of conventional histology followed that produce a fast specimen sample for examination or a permanent sample for further assessment. In brief the protocol for rapidly producing a specimen sample for examination entails quickly freezing the tissue, sectioning the tissue into thin slices, fixing the sections in formaldehyde, staining the sections, drying, and cover slipping the sections which are now ready for review. While the protocol is fast, the results are not permanent. So a second protocol is followed to produce a permanent sample, which will enable a more complete analysis. In order for fresh tissue sample to be preserved for future examination, fixation is needed and the resulted specimen is referred to as being fixed. Boiling an egg is an example of fixation, in which heat and chemistry stabilizes the organic material of the egg. Like the egg, a tissue specimen is hardened in order to provide microscopic information.

The permanent sample protocol consists of thawing the frozen tissue, dehydrating, fixing, embedding in paraffin wax, sectioning, deparaffinization of sections, hydrating, staining, dehydrating, drying, and placing a cover slip over the sample. However, this permanent protocol is extremely long and burdensome.

While conventional histology entails protocols to provide an easy to read tissue sample, the process is costly, as materials such as formaldehyde prove to be expensive, and time consuming, results are produced within a long time period.

Assortments of cytology and histology methods have been formed to provide manners of gaining information from cells.

U.S. Pat. No. 4,595,524 issued to Kin F. Yip on Jun. 17, 1986, and U.S. Pat. No. 4,741,898 issued to Arsum Malik on May 3, 1988, and U.S. Pat. No. 5,942,410 issued to Paul Pong-Shing Lam on Aug. 24, 1999, all show stains and processes for obtaining diagnostic information from tissues and cells, yet unlike the present invention these stains and protocols do not utilize a unique eosin/hematoxylin stain combination or inexpensive materials.

U.S. Pat. No. 6,459,805 issued to C. Patrick Reynolds on Oct. 1, 2003 shows fluorescence digital imaging microscopy system, yet unlike the present information requires expensive computer microscope technology.

In regard to the previous noted inadequacies of convention histology methods, there is a need for histology staining methods and protocols that reveal more information concerning chemical components and structure of cells, tissue, and organs; yet are also inexpensive and non-time consuming or cumbersome.

### SUMMARY OF THE INVENTION

In one phase, the present invention supplies a new solution that is effective in staining histological slides of cells, tissues, and organs. The solution comprises of a combination, of eosin and hematoxylin, where eosin is applied to a specimen first, and hematoxylin is applied as a second dye upon the specimen. This combination employed by the present invention, will bring forth new information through histological slides in teaching or studying, research, and diagnostic fields.

In a second phase, the present invention relates to two histological staining methods. In the methods provided by the present invention, the combination of eosin and hematoxylin is applied to a specimen to provide a stained cell or tissue sample. The present invention also supplies a method containing the procedure of staining the specimen with the combination solution noted previously to provide a stained specimen. The stained specimen can then as a result be further easily examined. One of the methods employed will rapidly produce a specimen sample for quick diagnostic review. A second method employed by the present invention will provide a permanent sample for future review.

### DETAILED DESCRIPTION OF THE PREFERRED EMOBODIMENTS

The present invention relates to a solution and method for staining histological slides of cells, tissues, and organs. More specifically, the present invention provides a solution that is useful in staining specimens that are generally colorless and flexible, and thus allows such specimens to provide ample information for scientific or diagnostic purposes when viewing under a microscope. The combination stain employed by the present invention includes eosin and hematoxylin, applied to the specimen in the respective order.

It has been established a method of using hematoxylin as a first dye, and eosin as a second dye when staining slides of cells, tissue, and organs. Yet, more information can be found from the tissue sample according to the present invention's combination stain and method. Combining eosin and hematoxylin, in this respective order, makes the stain of the present invention. The order of mixing the separate parts is significant and essential, because new information that will provide an improved understanding of cellular life in normal and sick organism. Upon using the present invention's stain and protocol, one can observe cell structures, molecules, and enzyme activity that are lost or not made present when using a conventional histological investigation solution and method of hematoxlin as a first dye and eosin as a second dye.

In relation to the second phase of the present invention, the stain solution expressed here is employed in an immensely enhanced method of staining tissue specimens that can be accomplished in a matter of 20-30 seconds as opposed to the regular 10-15 minutes of conventional histological staining methods. The rapid speed of the present invention proves to be beneficial for doctors in the surgical field that will need to make immediate decisions concerning nature and extent of surgery. The protocol of the present invention, because of its fast nature will allow surgeries to be finished 10-15 minutes faster. Compared to conventional staining methods, the present invention requires fewer materials, and the modest amount of materials utilized is relatively less expensive methods. For example, the use of alcohol for fixing purposes instead of formaldehyde not only kills most pathogens in the tissue but also is less expensive to buy and employ.

The tissue specimen to be stained is made frozen by known techniques. The frozen tissue specimen is then sectioned, sliced in order to provide a very thin specimen required for microscopy. After sectioning, the tissue is fixed to stabilize and preserve the tissue sample for future examination. The specimen is fixed by dipping the sectioned specimen 10 intervals in a watery 80% alcohol solution.

Next dipping the sectioned specimen 10 intervals in an eosin solution stains the eosinophillic substances of the section. The section is then removed from the eosin solution and dipped 10 intervals in tap water and dipped 20 intervals in a hematoxylin solution. After the basophilic structures have been stained by the hematoxylin solution, the section is dipped 10 intervals in tap water to complete the staining process. Due to the fact that hematoxylin has an affinity for negatively charged molecules and binds to acidic components of tissues; and eosin has an affinity for positively charged molecules and thus binds to basic tissue components; at this point of the staining procedure the tissue specimen is fixed and has a bluish/purple (acidic “basophilic” components of tissue) and pinkish/red (basic “acidophilic” components of tissue) coloring. The specimen is then air dried, mounted with a cover slip, and now ready to be read and analyzed. [Para 20]The aforementioned explained protocol of the present invention is particularly advantageous because the procedure is rapidly completed and permits rapid assessment of the sample. Immediate diagnostic information can be provided to a surgeon in the operating room. This is extremely beneficial in the medical field, especially for surgical doctors who often need to make immediate decisions concerning the nature and extent of surgery. Frozen sections can also allow analysis of small dispersible molecules or of enzyme activity whose existence would be lost during chemical fixation. Yet, while it permits a rapid assessment of the sample, the preparation is not permanent and has a limited life span. Also, a sample prepared by these rapid protocol posses a less appealing appearance then well fixed specimens. In order to have a more precise microscopic analysis, as well as a permanent preparation of the original sample, a second method is pursued. In this method, the frozen section of the previously explained fast protocol for frozen sections is thawed, fixed, dehydrated, penetrated with solvents such as alcohol, embedded in paraffin and sectioned. The sections are then deparaffinized so that no paraffin wax is visible or remaining on the sectioned samples. The sections are next placed in a watery 80% alcohol solution.

Next dipping the sectioned specimen 10 intervals in an eosin solution stains the eosinophillic substances of the sections. The section is then removed from the eosin solution and dipped 10 intervals in tap water and afterward dipped 20 intervals in a hematoxylin solution. After the basophilic structures have been stained by the hematoxylin solution, the section is dipped 10 intervals in tap water. Once again the section is dipped for 10 intervals in the eosin solution and subsequently dipped for 10 intervals in tap water. The section is removed from the tap water, dipped for 20 intervals in the hematoxylin solution, and once again dipped 10 intervals in tap water. Next, the section is dipped for ten intervals in the eosin solution and dipped for 10 intervals in tap water to bring the staining process to a conclusion. The specimen is then air dried, mounted with a cover slip, and now prepared to be read and analyzed. The slide should show portions of the specimen stained blue and other components stained in hues of pink and red. This protocol provides a permanent microscopic section preparation, without extreme length of time or cumbersomeness ability.

1. A method for staining, comprising:
first immersing a sample in an eosin solution; and
then immersing the sample in a hematoxylin solution.

2. The method of claim 1, further comprising fixing the sample in a watery alcohol solution.

3. The method of claim 2, wherein the watery alcohol solution is 80 percent alcohol.

4. The method of claim 1, wherein the sample is immersed in the eosin solution for ten intervals.

5. The method of claim 1, further comprising immersing the sample in tap water.

6. The method of claim 1, wherein the sample is immersed in the hematoxylin solution for 20 intervals.

7. The method of claim 5, wherein the sample is immersed in tap water for ten intervals.

8. The method of claim 1, further comprising air drying the sample.

9. The method of claim 1, further comprising immersing the sample in an eosin solution after first immersing the sample in the eosin solution and secondly immersing the sample in the hematoxylin solution.

10. The method of claim 9, wherein the second immersion of the sample in eosin is for ten intervals.

11. The method of claim 9, further comprising immersing the sample in tap water after the second immersion of the sample in eosin.

12. The method of claim 11, wherein immersing the sample in tap water after the second immersion is for ten intervals.

13. A method for staining, comprising:
first immersing a sample in an eosin solution;
then immersing the sample in a water solution;
then immersing the sample in a hematoxylin solution;
then immersing the sample in a water solution;
then immersing the sample in an eosin solution;
then immersing the sample in a water solution; and
then air drying the sample.
